(19) United States
(12) Patent Application Publication
(10) Pub. No.: US 2004/0005694 A1
(43) Pub. Date: Jan. 8, 2004

(54) Light decontamination of fermentation media

(76) Inventor: Herbert Lutz, Belmont, MA (US)

Correspondence Address: MILLIPORE CORPORATION, 290 CONCORD ROAD, BILLERICA, MA, 01821, US

(21) Appl. No.: 10/429,902

(22) Filed: May 5, 2003

Related U.S. Application Data
(60) Provisional application No. 60/378,779, filed on May 8, 2002.

(51) Int. Cl. C12P 1/00; C12N 7/02; C12N 13/00

(52) U.S. Cl. 435/239; 435/41; 435/173.1

(57) ABSTRACT

Fermentation media, employed for its ability to grow recombinant mammalian cells to high densities and abet their expression of recombinant protein drug products at high titers, are rendered free of active viruses by treatment with low levels of light, levels at which the essential media properties are retained.

This invention relates to methods of viral inactivation of aqueous solutions used in the production of biopharmaceuticals from mammalian expression systems. More particularly, the invention contemplates methods of inactivating viruses in fermentation media used to propagate characterized cell lines.

### BACKGROUND OF THE INVENTION

Fermentation media is widely used in the batch production of recombinant pharmaceuticals from mammalian expression systems. This production method has been growing steadily and now represents a significant fraction of all drug product manufacture. Viral contamination of the media can lead to host cells infection and viral titer amplification. High virus titters can overload the virus clearance capabilities of the purification process and virus assay methods are slow and limited in their ability to detect all contaminants. This raises concern over the safety of the purified drug product. Even when contamination is detected, significant costs are incurred in scrapping batches and plant shutdowns to sanitize the entire system.

Bioreactor contamination by viruses is a growing concern. Recently, a number of contamination incidents have been reported. While cGMP and viral assays on media components are employed to reduce contamination, there is a substantial interest in finding additional methods to ensure that media remains free of the viable infectious agent.

### SUMMARY OF THE INVENTION

Fermentation media used for mammalian expression systems are freed of infectious viral agents using light treatment. Viruses are inactivated and the fermentation media retains its essential properties of growing cells to high densities and abetting the expression of recombinant drug products.

### IN THE DRAWINGS

### DETAILED DESCRIPTION OF THE SPECIFIC EMBODIMENTS

In accordance with the subject invention, methods and compositions are provided involving the inactivation of infectious viruses from fermentation media, while retaining the essential properties of the media, to provide media which is free of viral infection.

Tissue culture media, used in growing mammalian cells, is introduced into the fermentor as a liquid solution. A wide variety of media, e.g. Dulbecco's Modified Eagle's Medium (DMEM), are employed containing a multitude of components essential for cell growth and product protein expression. These components include salts, sugars, buffering agents, amino acids, vitamins, surfactants, and proteins. The aqueous medium is contacted with a virus deactivating amount of light for a short time under mild conditions and the aqueous medium removed from the light treatment, and may be subject to further treatment such as contact with a reducing agent. The resulting composition is freed of viable virus.

The aqueous media will generally have total inorganic salts at around the isotonic osmolarity of 9000 mg/L. The pH will normally be close to the physiologic pH 7. Amino acids will range from 500 to 1200 mg/L. Other components which may be present in the medium include trace metals, lipids, protein stabilizers, cell wall stabilizers, anti-oxidants, nutritive proteins, sugars, and the like.

The subject method is effective with a wide variety of virus families including those with RNA or DNA genomes and those with or without a lipid envelope. Of particular interest are the non-enveloped, DNA genome parvoviruses which have been generally accepted as highly resistant to inactivation and which have been known to contaminate cell cultures with adverse results.

In decontaminating the biological product, the medium may be contacted with the light under a variety of conditions. Generally, exposure of the media to light in a static exposure is not be employed but may be used if desired. Various techniques for contacting the medium with light may include pumping the medium through a chamber containing the light such as a flowing channel, employing a thin film such as by flowing it in a thin stream across a flat plate, by using a falling film or by using rotating vessels, e.g., rotating bottles, or spraying the medium in a chamber filled with light. The dose of light will generally be from about 0.1-10 J/cm2. The atmosphere of the chamber may be formed of inert gasses, such as nitrogen, or may be air. The temperature may be maintained from about 4° to 37° C., and more usually from about 25° to 37° C. The exposure time will vary widely, depending upon the ability of the fluid to transmit light, the depth of the fluid to be penetrated by the light, the nature of the suspected contamination, and the light intensity. Generally employing about 0.1 hour to 8 hours of light is sufficient to eliminate contamination. In another embodiment, 0.2 to 4 hours is sufficient.

After the medium has been treated, it may be further treated with reducing agents to ensure the absence of any free radicals. Small amounts of ascorbic acid, glutathione, sodium thionite, or the like, namely reducing agents which are physiologically acceptable, may be employed. The amounts will generally range from about 20 μg to about 2 mg/ml.

After the media has been decontaminated, it may then be introduced into the fermentor for cell growth and protein expression. Alternatively, one may employ a viral filter such as a VIRESOLVE® or RETROPORE™ filter available from Millipore Corporation of Bedford, Mass. to remove the inactivated viral component.

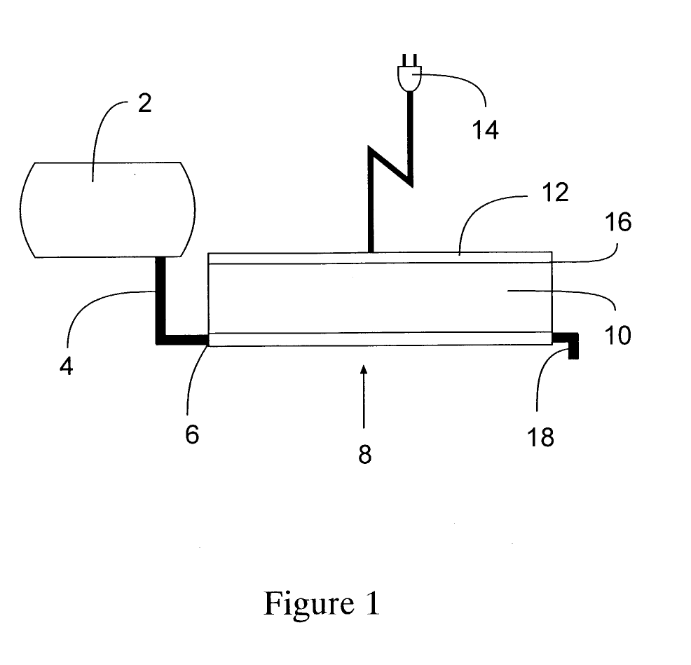

FIG. 1 shows a preferred embodiment of a system of the present invention. The media supply 2 is connected to a conduit 4 that connects with the inlet 6 of the light treatment device 8. The treatment device in this embodiment is a chamber 10. A light source 12 is mounted against or if desired in the chamber 10 with an external power supply 14. As shown, the light source 12 is mounted adjacent the chamber 10 and provided to the chamber 10 via a window 16 which is typically a quartz or glass material. The media enters inlet 6 and is exposed to the light supplied to the chamber 10 through the light source 12 for a period of time sufficient to inactivate the viral components. The treated media is then decanted through outlet 18.

The light spectrum used for inactivation can include single or broad spectrum wavelengths containing light within the UV spectrum (200-400 nm). The light can be applied either as a continuous process or in a pulsed process so long as the resultant dose has a delivered energy dose ranging from 0.1-10 J/cm².

The following examples are offered by way of illustration and not by way of limitation.

### EXAMPLE #1

The following is a description of an embodiment of the present invention:

Light Exposure System:

The system accommodates two vessels for light exposure. To prevent the reaction of light with non-biological components of the system, all the system components which come into contact with light are made with the non-absorbing materials such as glass, Teflon®, polyalkanes, or stainless steel. Compressed air (3.5 kg/cm²) is introduced into the system through a pressure regulator set at 141 g/cm² and is filtered through two microfilters (Aervex™ filters of Millipore Corp., Bedford, Mass.) in series. Each microfilter is absolutely retentive of bacteria and viruses.

This light is directed into a contacting device at room temperature.

Light Generation and Monitoring:

Light with wavelengths ranging from 200 nm to 400 nm is generated using fluorescent bulbs operating with a regulated constant voltage power supply.

Exposure of Product to Light:

Fermentation media samples are spiked with 10⁹ pfu/ml of porcine parvovirus, a 18-26 nm non-enveloped, single stranded DNA virus of the parvoviridae family obtained from the American Tissue Type Collection (ATCC) in Rockville, Md. 250 ml aliquots of the contaminated product to be exposed is placed in glass bottles. The vessels are connected to the exposure system and contacted with the light. The product is exposed for 10 minutes and receives approximately a delivered energy dose of about 1 J/cm².

The light-treated media and initial spiked media is then assayed for the presence of active virus involving the serial dilution and plating of ESK-4 cells in quadruplicate on 96 well plates. Plates were observed after 10 days for the presence of cytopathic effects and a 50% infectious dose (TCID₅₀) calculated using the Reed-Muench formula.

### EXAMPLE #2

The following is a description of a contemplated experiment:

Light exposure system: Xenon gas lamps in a quartz tube.

The vessels were flushed with 1 L WFI. A 1 Liter bag of media was pumped through system at 0.25 Lpm as an untreated control. The system was then flushed with 1L WFI and a fresh 1 L bag of media containing a 10 ml spike of Porcine Parvovirus at roughly 105 PFU/ml was attached to the system. The sample was processed at 0.25 Lpm with broad spectrum light pulses at 1.5 Hz. At this rate, each fluid element sees 4 flashes as it traverses the treatment chamber. The 10 ml of treated media was then sampled. Samples of spiked feed and treated media were diluted and inoculated on porcine testicle cells (from ATCC) for assay by plague formation. No viruses were detected in the 10 ml of treated media. Accounting for sampling error in the treated media by the Poisson distribution, a >4.3 LRV in virus was observed.

The subject invention provides for a rapid, reliable, economic procedure for decontaminating fermentation media, freeing the products of viruses, so that the products may be used without the risk of viral contamination of the bioreactor. Furthermore, the media retains its ability to grow mammalian cells to high densities and support the expression of recombinant protein drug products in high concentrations. The method is easy to perform, large amounts of product can be treated, and the system is free of production of products that may have deleterious biological effects.

Although the foregoing invention has been described in some detail by way of illustration and example for purposes of clarity of understanding, it will be obvious that certain changes and modifications may be practiced within the scope of the appended claims.

1. A method for freeing fermentation media of viable viruses while retaining the essential characteristics of the media, said method comprising:
contacting said media with a virus inactivating amount of light under mild conditions for a sufficient time to inactivate all viruses present; and
isolating the media, rendered free of active viruses.

2. A method according to claim 1 wherein said contacting is at a temperature in the range of 4° to 37° C. and at light concentration of from 0.1-10 J/cm².

3. A method according to claim 1 wherein said biological product is contacted as a thin static film or in a flowing channel.

4. A method according to claim 1 wherein the contacting is for a duration of 0.1 hour to 8 hours.

5. A method according to claim 1 wherein biological product is contacted with light selected from the group consisting of UV, pulsed white light and combinations thereof.

6. A method according to claim 1 wherein an aqueous solution of a biological product is contacted with light.
